(19) United States
(12) Patent Application Publication
(10) Pub. No.: US 2004/0137626 A1
(43) Pub. Date: Jul. 15, 2004

(54) AAV ITR-mediated modulation

(75) Inventors: Thomas E. Wagner, Greer, SC (US); Xianzhong Yu, Mauldin, SC (US)

Correspondence Address: FOLEY AND LARDNER, SUITE 500, 3000 K STREET NW, WASHINGTON, DC, 20007, US

(73) Assignee: Greenville Hospital System

(21) Appl. No.: 10/669,641

(22) Filed: Sep. 25, 2003

Related U.S. Application Data
(60) Provisional application No. 60/413,450, filed on Sep. 26, 2002.

(51) Int. Cl. A61K 48/00; C12N 15/861; C12N 15/86; C07H 21/04

(52) U.S. Cl. 435/456; 514/44; 536/23.2

(57) ABSTRACT

The present invention relates to a stabilized nucleic acid that kills tumor cells and methods for producing the same. Specifically, the nucleic acid drug comprises pairs of viral inverted terminal repeat hairpin loops which elicit cell apoptosis. The present invention also provides methods for making such a stabilized nucleic acid drug as well as methods for targeting the drug to a cell nucleus or genome. Accordingly, the nucleic acid drug of the present invention is useful for inducing apoptosis in cells, especially those lacking p53, such as cancer cells.

### CROSS-REFERENCE TO RELATED PATENT APPLICATIONS

This U.S. Non-Provisional application claims priority to and benefit of U.S. Provisional application No. 60/413,450, filed on Sep. 26, 2002, which is incorporated herein by reference in its entirety.

### FIELD OF THE INVENTION

The present invention relates to a nucleic acid drug that kills tumor cells and methods for producing the same.

### BACKGROUND

The adeno-associated virus inverted terminal repeat sequence (“AAV-ITR”) is known to induce apoptosis in tumor cells which lack p53, a protein which controls cell division. See de la Maza & Carter, J. Natl. Cancer Institute, 67(6): 1323-1326, 1981, and Raj et al., Nature, 412: 914-917, August, 2001. de la Maza & Carter showed that adeno-associated viruses and their DNA inhibited tumors in Syrian golden hamsters. Similarly, Raj et al., demonstrated that virus-, or virus-like-particles containing inverted terminal repeats induced apoptosis in cells lacking p53.

It is well known that p53 is inactivated in almost all types of human cancers. For a review, see Vogelstein & Kinzler, Nature, 412: 865-866, August, 2001. However, it also is known that p53 prevents cellular DNA replication and division in the presence of damaged DNA (i.e., p53 prevents cells from progressing from their “resting stage” to DNA-replication or from progressing from the “second resting stage” to nuclear division). For instance, cellular proliferation of cells containing normal p53 pauses during mitosis so that foreign or damaged DNA can be repaired or removed from that cell.

In the absence of p53, cell mitosis is not interrupted and in cells with damaged DNA, the cells proceed to replicate, divide and proliferate. Thus, cells with damaged DNA and without functional p53 arrest transiently and die. This observation suggests that failure to sustain mitotic arrest in the presence of damaged DNA leads to cell death through apoptosis.

It is believed that cells incorrectly identify the conformational structure of AAV ITR polynucleotides, such as hairpin loops, as that of damaged DNA. Accordingly, the presence of AAV ITR polynucleotides causes the cellular machinery to act as though the cell contains damaged DNA. It follows that in the presence of AAV ITR, cells lacking functional p53 proteins undergo apoptosis and die. Accordingly, AAV ITR can be used to selectively elicit apoptosis in certain cell types, i.e., cancer cells.

However, “unprotected” and free DNA molecules, i.e., nucleic acids that are not packaged into a viral particle, are unstable and susceptible to degradation if introduced into a cell. Furthermore, no therapeutic composition comprising a stable AAV element complex that induces apoptosis of cancer cells has been described. Accordingly, described herein is a novel, highly stable “nucleic acid drug” comprised of AAV ITR sequences that can be used to induce cell death. Moreover, the present methods avoid chemical synthesis of individual AAV ITR sequences, which prove to be impractical and not cost-effective.

### SUMMARY

Accordingly, described herein is a nucleic acid drug and methods for making that drug.

In one aspect of the present invention, an isolated nucleic acid drug (“drug 1”) comprising four pairs of hairpin loops is envisioned, wherein any or all of the pairs of hairpin loops are capable of inducing cell apoptosis. In one embodiment, the isolated nucleic acid drug is comprised of DNA or RNA. In another embodiment, the nucleic acid of the nucleic acid drug is DNA. In yet another embodiment, the nucleic acid drug comprises the DNA sequence of an AAV ITR. In a more preferred embodiment, the AAV ITR sequence has the sequence of SEQ ID NO. 1. In yet another embodiment, the isolated nucleic acid drug further comprises at least one nuclear localization signal peptide. In a preferred embodiment, the nuclear localization signal peptide is associated with said nucleic acid drug via a PNA-clamp, which comprises a biotin molecule. The biotin molecule is bound to a streptavidin molecule that comprises at least one nuclear localization signal peptide. In one embodiment, the PNA-clamp anneals to a target sequence present in said nucleic acid drug. In a preferred embodiment, the target sequence is located in a “spacer” portion of the nucleic acid drug.

In a preferred embodiment, the nuclear localization signal peptide is selected from the group consisting of an SV40 nuclear localization signal peptide, a poly-L-lysine, an antennapedia peptide, a TAT peptide, a c-myc peptide, a VirD2 peptide, a nucleoplasmin peptide, an ARNT derived peptide and an M9 domain peptide.

The invention also envisions an apoptosis-inducing formulation comprising a nucleic acid drug which comprises four pairs of hairpin loops. In another embodiment, the formulation further comprises a DNase inhibitor.

In another aspect, the present invention contemplates a plasmid that comprises a construct for making the nucleic acid drug. The construct comprises in 5′- to 3′-order (i) a first arm polynucleotide sequence, (ii) a spacer polynucleotide sequence, and (iii) a second arm polynucleotide sequence, wherein the second arm polynucleotide sequence is the complement of the first arm polynucleotide sequence and wherein the second arm polynucleotide sequence is in the opposite orientation of the first arm. That is, the orientation of the second arm is such that upon dissociation, its two single strands can anneal with their complementary partner sequences present in the first arm. In a preferred embodiment, the construct is flanked by the same or different restriction sites. In another preferred embodiment, the plasmid comprises at least two of constructs. In another embodiment, the plasmid comprises at least four constructs, at least six constructs, at least ten constructs or more than twelve constructs.

In yet another embodiment, each construct of any one of these plasmids can be physically separated from each of the other constructs by exposing the plasmid to one or more restriction enzymes that recognize the restriction site sequences that flank each of the constructs.

The present invention also contemplates a cell comprising any one of these plasmids. In a preferred embodiment, the cell is a bacterial cell, mammalian cell, viral cell, yeast cell or fungal cell. In a preferred embodiment, the cell is an E. coli cell.

The present invention also encompasses a nucleic acid drug (“drug 2”), comprising (i) a PNA-clamp comprising a biotin molecule; (ii) a streptavidin molecule comprising at least one nuclear localization signal peptide; and (iii) an AAV ITR polynucleotide with a 5′ end and a 3′ end, wherein said PNA-clamp is hybridized to the 3′-end of said AAV ITR polynucleotide, wherein said AAV ITR folds into a pair of hairpin loops, wherein the biotin molecule is bound to the streptavidin molecule, and wherein the nucleic acid drug targets the nucleus or genome of a cell.

In a preferred embodiment, the nuclear localization signal peptide is selected from the group consisting of an SV40 nuclear localization signal peptide, a poly-L-lysine, an antennapedia peptide, a TAT peptide, a c-myc peptide, a VirD2 peptide, a nucleoplasmin peptide, an ARNT derived peptide and an M9 domain peptide. In yet another preferred embodiment, the AAV ITR polynucleotide comprises the sequence described in SEQ ID NO. 1.

The invention also envisions a cell comprising the nucleic acid drug of either or both drug 1 and/or drug 2. In one embodiment the cell is a bacterial cell, mammalian cell, viral cell, yeast cell or fungal cell. In a preferred embodiment, the cell is an E. coli cell.

In yet one other aspect of the present invention, a method (“method 1”) for producing a nucleic acid drug is provided. This method comprises transforming a cell with, for example, any one of the plasmids described herein, incubating the cell under conditions that promote cell growth, isolating the plasmid DNA from the culture, adding at least one restriction enzyme to the isolated plasmid DNA to generate discreet constructs, and denaturing the discreet constructs to produce single-stranded nucleic acids, wherein the single-stranded nucleic acids hybridize to sequences present in their own strand as well as to complementary sequences in other single strands to produce a nucleic acid drug. In one embodiment, the cell is a bacterial cell. In another embodiment, the cell is an E. coli cell.

In yet another embodiment, the method further comprises adding a PNA-clamp to the nucleic acid drug. In one embodiment, the PNA-clamp is capable of binding to another molecule that comprises at least one nuclear localization signal. In a preferred embodiment, the PNA-clamp comprises a biotin molecule bound to a streptavidin molecule that comprises at least one nuclear localization signal peptide, wherein the PNA-clamp is hybridized to a sequence present in a part of the nucleic acid drug. In a preferred embodiment, the PNA-clamp is hybridized to a nucleic acid sequence present in the spacer portion of the nucleic acid drug.

Another method (“method 2”) produces a nucleic acid drug using the polymerase chain reaction to amplify a polynucleotide sequence that comprises (i) a first arm polynucleotide sequence, (ii) a spacer polynucleotide sequence, and (iii) a second arm polynucleotide sequence. In one embodiment, the second arm polynucleotide sequence is the complement of the first arm polynucleotide sequence and is oriented in the opposite position to the first arm. Each “arm” can be the sequence of the AAV ITR polynucleotide or its complement sequence. However, any sequence that folds into the conformation desired by the present invention can be used as an “arm” according to the described methods. Thus, the polynucleotide sequence may comprise, for instance, in 5′ to 3′ orientation: a 145 bp of AAV ITR sequence, a 100 bp spacer sequence, and an oppositely-oriented (i.e., complementary) 145 bp AAV ITR sequence. However, the length of any part of the polynucleotide is not limited to 145 bp and 100 bp elements. An “arm” that folds into a desired conformation, such as into the two hairpin loops of the AAV ITR molecule, known as the “kissing ears” configuration, may be at least 50 bp, at least 100 bp, at least 150 bp, at least 200 bp, at least 250 bp, at least 300 bp, at least 350 bp, or more than 400 bp.

Method 2 entails isolating the amplification products from the polymerase chain reaction, denaturing the amplification products to form single strands and allowing the single strands to reanneal into hairpin-stem loop structures, wherein at least some of the reannealed structures comprise four pairs of hairpin loops, wherein the reannealed structures are the nucleic acid drugs.

In a preferred embodiment, the first arm polynucleotide sequence has the sequence described in SEQ ID NO. 1, and the second arm polynucleotide is the complement of the sequence described in SEQ ID NO. 1.

The invention also provides a method (“method 3”) for delivering a nucleic acid drug to the genome of a cell, comprising providing at least one target cell; and introducing at least one nucleic acid drug of the present invention to the target cell. In a preferred embodiment, the nucleic acid drug enters the target cell and induces cell apoptosis. In another embodiment, the nucleic acid drug is directed to the cell nucleus or genome by virtue of an associated nuclear localization signal. In one embodiment, the cell is a eukaryotic or prokaryotic cell. In another embodiment, the cell is a disease cell. In yet another embodiment, the target cell does not contain a functional p53 protein. In a preferred embodiment, the target cell is a cancer cell.

The present invention also contemplates a method (“method 4”) for inducing apoptosis in tumor cells of a living animal. This method comprises introducing at least one nucleic acid drug of the present invention into an animal, wherein the nucleic acid drug enters a cell lacking a functional p53 protein and induces apoptosis of the cell. In a preferred embodiment the nucleic acid drug is introduced into the animal by intravenous injection, topical application, aerosol, through the nasal mucosa, rectally, or orally. In another embodiment, the animal is a mammal. In a preferred embodiment, the mammal is a mouse, rat, rabbit, cat, dog, pig, cattle, monkey, or human. In a more preferred embodiment, the mammal is a human. In another embodiment, the animal is a bird or reptile.

### BRIEF DESCRIPTION OF THE DRAWINGS

FIG. 1 illustrates the folded conformation of a single AAV ITR nucleic acid sequence.

FIG. 2 is a schematic of a folded conformation of a single AAV ITR nucleic acid. The linear sequence represents one strand of an AAV ITR “arm”. A plasmid of the present invention may comprise in 5′- to 3′ direction, a first arm, a spacer and an inverse complement of a second arm (see FIG. 3).

FIG. 3 is a schematic illustrating the relationship between a linear, double-stranded AAV ITR clone, and the complex formed after denaturation and reannealing of the AAV ITR clone. The clone may reside in a plasmid or may be a template for PCR. In the former case, the clone can be released from the plasmid by digesting the plasmid with enzymes that recognize restriction sites located upstream of “Arm 1” and downstream of “Arm 2.” The double-stranded molecule can then be heat-denatured and separated into single strands which form the resultant complex. In the latter case, the clone can be amplified by PCR directly and, similarly, heat-denatured to produce such a complex.

### DETAILED DESCRIPTION OF THE PREFERRED EMBODIMENTS

The present invention relates to the use of a stabilized nucleic acid drug, which comprises pairs of inverted terminal repeat hairpin loops, to elicit cell apoptosis. The present invention also provides methods for making such a stabilized nucleic acid drug as well as methods for targeting the drug to a cell nucleus or genome. Accordingly, the nucleic acid drug of the present invention is useful for inducing apoptosis in cells, especially those lacking p53.

According to the present invention, the nucleic acid drug is a complex of complementary single stranded DNA or RNA molecules, which, when annealed to one another, create a highly stable nucleic acid complex. The stabilized nucleic acid drug comprises a complex of inverted repeat nucleic acids that form hairpin loops and which induce cell death in p53-negative cells. As discussed above, cells with damaged DNA that lack functional p53 protein, or which do not express the p53 protein, are unable to prevent cell proliferation and they ultimately die from apoptosis. Accordingly, the hairpin loops of the stabilized nucleic acid drug are important in eliciting apoptosis in such cell types.

The inverted repeat sequence that folds to form these hairpin loops can be an adeno-associated virus inverted repeat nucleic acid sequence. However, any nucleic acid having features corresponding to those of an AAV ITR sequence may be used. For instance, it is only necessary that a nucleic acid contains regions that enable self-complementarity to bring about folding into double-stranded hairpin loops. Typically, a single-stranded AAV ITR nucleic acid contains 145 nucleotides which folds up on itself to produce a partially-double-stranded “Y”-like structure. The “V” of the “Y” comprises double-stranded hairpin loops. Each of these short hairpin loops comprises unpaired nucleotides which are complementary to one another. It is believed that these complementary nucleotides are capable of forming hydrogen bonds that create a close spatial relationship between the two stem-loops in the Y-shaped structure. These loops are “kissing ears.” It is the presence of these “ears” that is thought to induce a cell's response to damaged DNA.

An AAV ITR sequence may have a sequence such as that described in SEQ ID NO. 1:

| | |
|---|---|
| 5′-ttggccactccctctctgcgcgctcgctcgctcactgaggccgg | (SEQ ID NO. 1) |
| gcgaccaaaggtcgcccgacgccgggctttgcccgggcggcctcagtgagcgagcgagcgcgcagagag | |
| ggagtggccaactccatcactaggggttcct-3′. | |

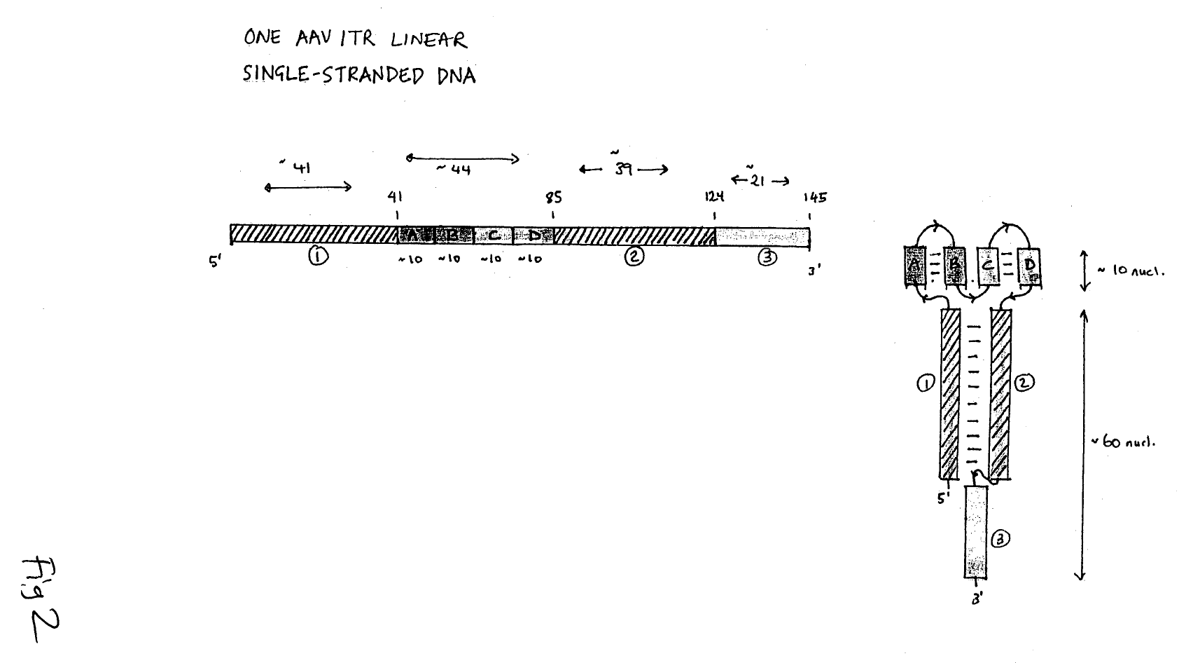

However, production of such a small molecule (the double-stranded stem of the folded “Y” structure is only about 40 bp in length) by chemical synthesis is not practical or cost-effective, and the small size alone makes the short polynucleotide difficult to manipulate and create individual ITR molecules. The present invention provides a novel method for making nucleic acid kissing ears in a rapid and efficient fashion, and moreover, potentiate a cell's “DNA damage” response mechanism. The latter is possible because each of the stabilized nucleic acid drugs of the present invention can be made to contain four pairs of kissing ears, thereby increasing the relative concentration of these molecules per cell. Such a complex is shown in FIG. 2.

Each of the four “arms” of the complex is a folded AAV ITR nucleic acid and from each arm protrudes a pair of kissing ears. A spacer nucleic acid of any length can be used to separate two arms from the other two arms to produce an elongated “X” structure. The spacer nucleic acid also serves to assist in amplification and recombinant manipulation of the nucleic acid, by simply increasing the length of the nucleic acid that one has to work with. The spacer sequence also may be a site into which a “target site” may be introduced to which a complementary polynucleotide can anneal. Accordingly, the entire “four-arm” complex can be made by the polymerase chain reaction (PCR) or by cloning a double-stranded nucleic acid into a plasmid that is used to transform cultures of cells.

Thus, one method of the present invention involves cloning two “arm” sequences, separated by the spacer nucleic acid, into a plasmid, vector or other such nucleic acid carrier. Accordingly, the cloned linear, double-stranded molecule comprises, in 5′- to 3′-orientation, (i) a double-stranded “first arm” sequence, (ii) a double-stranded spacer sequence, and (iii) a double-stranded “second arm” sequence. The sequence of the second arm is in opposite orientation to the sequence of the first arm. In such orientation, sequences in the first arm will anneal or hybridize to sequences of the second arm when the double-stranded DNA is separated into single strands.

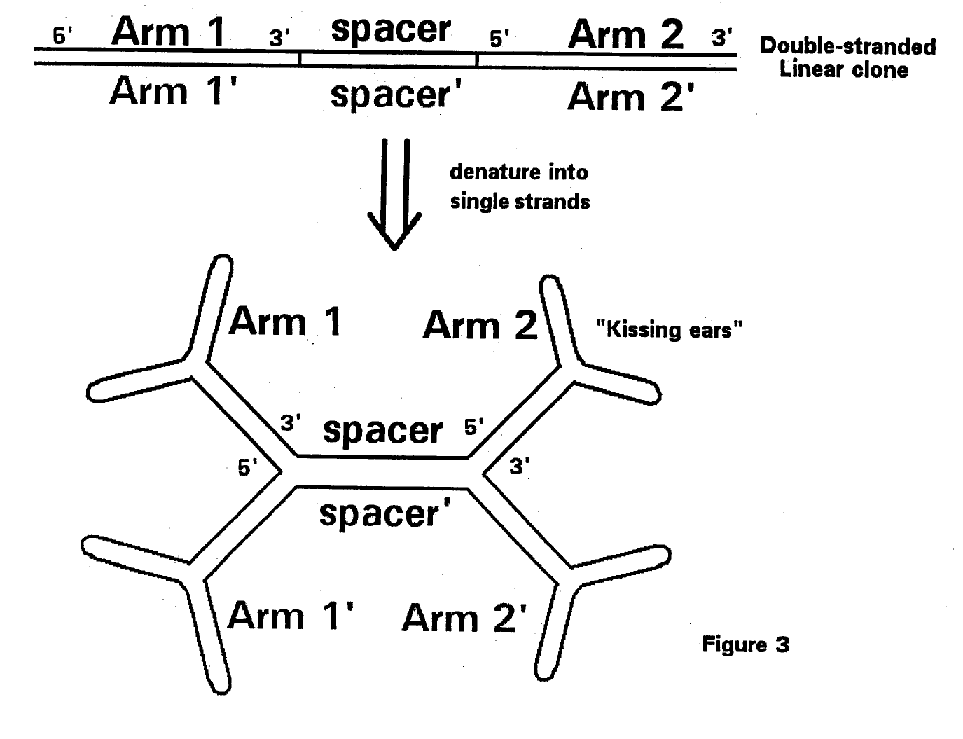

Accordingly, each strand of each arm comprises complementary sequences that can anneal to one another to form the kissing ears, as well as sequences that enable the first arm to anneal to the second arm. Restriction enzyme recognition sites can be inserted into the plasmid upstream of the first arm and downstream of the second arm, allowing the entire construct to be cut from the plasmid with ease. The resultant double-stranded construct comprises a “[first arm]-[spacer]-[second arm]” structure. See FIG. 3.

The double-stranded construct can then be denatured, e.g., by heating the molecule so that it dissociates into single stranded molecules. Since the “sense” strand and the “antisense” strand of each arm are also complementary to each other at their “spacer” regions, four arms are provided by the cloned fragment. Each of those arms is capable of folding into the desired kissing ears to produce the nucleic acid drug (which comprises a first arm sense strand, a first arm antisense strand, a second arm sense strand and a second arm antisense strand). See the bottom half of FIG. 3, which illustrates such a hairpin loop conformation. The sequence of the arms may be that of the AAV ITR, but any nucleotide sequence that can fold into a similar hairpin loop/multiple kissing ear structure can be used. As mentioned, the spacer arm can be of any length or sequence, and as discussed below, the spacer is also useful as a target site to which other DNA-bearing molecules can be attached.

Thus, the cloned nucleic acid drug comprises a first AAV ITR arm of 145 bp, a spacer sequence and, in opposite orientation, a second AAV ITR arm of 145 bp. The spacer sequence may be about 20 bp, 25 bp, 50 bp, 75 bp. 100 bp, 125 bp, 150 bp, 175 bp, 200 bp, 225 bp, 250 bp, 300 bp, or more than 300 bp in length. The entire construct is flanked by the same or different restriction recognition sites. Thus, a four-hundred or so base pair clone can be amplified by PCR and also cloned directly into a plasmid with ease. Furthermore, several of such constructs, each separated by the same or different restriction sites, can be cloned into a plasmid, thereby increasing the number of nucleic acid drugs that can be formed upon denaturation and annealing.

Bacterial cells, such as E. coli can be transformed with the plasmid and used to amplify the plasmid clones whenever necessary, by simply growing liters of cultured cells. Other cells, such as yeast, viral and mammalian cells can be used for the same purpose. The cells can then be pelleted, lysed and the plasmid DNA isolated. By then exposing the isolated plasmid DNA to restriction enzymes that recognize the restriction sites which separate the individual nucleic acid drug constructs, one may generate preparation of double-stranded DNA pre-drugs. The preparation may then be heat-denatured to produce the single-stranded molecules that fold and anneal to one another to produce the apoptosis-inducing stabilized nucleic acid drug with four pairs of kissing ears.

The methods described herein thus provide a convenient and efficient way to produce a nucleic acid drug that is effective in inducing cell apoptosis. One simply need grow a culture of plasmids containing at least one nucleic acid drug construct, and then simply digest and denature the isolated constructs to produce the highly stable nucleic acid drug. There is no need to package the construct into a viral particle.

The formed drug may be formulated with other compounds, such as DNase inhibitors, before being introduced into an animal. The nucleic acid drug may be introduced into a mouse, rat, rabbit, cat, dog, pig, cattle, monkey, bird, reptile, or human. The nucleic acid drug may be introduced by intravenous injection, topical application, aerosol, through the nasal mucosa, rectally, or orally.

Furthermore, the nucleic acid drug may comprise factors that direct the drug to a cell nucleus or cell genome. For instance, the nucleic acid drug may comprise at least one nuclear localization signal peptide (“NLS”) which target genetic material. Examples of NLS peptides include, but are not limited to an SV40 nuclear localization signal peptide, a poly-L-lysine, an antennapedia peptide, a TAT peptide, a c-myc peptide, a VirD2 peptide, a nucleoplasmin peptide, an ARNT derived peptide and an M9 domain peptide. A spacer sequence of the formed nucleic acid drug may be engineered to comprise a target site recognized by a molecule that comprises one or more NLSs. In this regard, the spacer sequence may be made to comprise a target site recognized by a complementary region of a nucleic acid PNA-clamp, which, through a biotin-streptavidin linkage, carries one or more NLS peptides. An example of such a target site is ggaggggtggagagagagagagaga (SEQ ID NO. 2) to which the PNA-clamp anneals. A PNA (“peptide nucleic acid”) hybridizes to single- and double-stranded nucleic acids. PNA (Gene Therapy Systems, San Diego, Calif.) are nucleic acid analogs in which the entire deoxyribose-phosphate backbone has been exchanged with a chemically different, but structurally homologous, peptide backbone containing 2-aminoethyl glycine units. Unlike DNA, which is highly negatively charged, the PNA backbone is neutral. Therefore, there is much less repulsive energy between complementary strands in a PNA-DNA hybrid than in the comparable DNA-DNA hybrid, and consequently the PNA-DNA hybrid, for example, is much more stable. PNA can hybridize to DNA in either a Watson/Crick or Hoogsteen fashion (Demidov et al., Proc. Natl. Acad. Sci. U.S.A. 92:2637-2641, 1995; Egholm, Nature 365:566-568, 1993; Nielsen et al., Science 254:1497-1500, 1991; Dueholm et al., New J. Chem. 21:19-31, 1997). See also U.S. Pat. No. 6,165,720. Accordingly, the PNA-clamp may also further stabilize the nucleic acid drug.

The formed nucleic acid drug may comprise one or more of such “PNA-clamp-NLS peptide” moieties. The target sequence or sequences recognized by the PNA-clamp may be incorporated into any part of the nucleic acid drug, not only in the spacer sequence, so long as binding of the PNA-clamp does not mask or destroy the conformation of a pair of kissing ears. To that end, it is not necessary that a PNA-clamp per se be used to associate NLS peptides with the nucleic acid drug. It is only necessary that some moiety contain a nucleic acid sequence that is complementary to a portion of the nucleic acid drug and that the moiety comprises at least one NLS peptide.

Furthermore, it is not necessary that a spacer sequence be included in the stabilized nucleic acid drug complex. The drug may simply comprise four hairpin loops that are associated together by annealing of stem-to-stem sequences present in the remaining portion of the AAV ITR sequences. Accordingly, these AAV ITR stem sequences may be engineered to contain target sequences like those described above and moieties such as the PNA-clamp hybridized thereon. Thus, the present invention envisions the annealing of nucleic acid sequences between discreet single-stranded molecules, as well as to the annealing of sequences to sequences present within the same single-stranded molecule.

The AAV ITR sequence, such as that described in SEQ ID NO. 1, can be made by overlapping oligonucleotides encoding parts of the entire sequence. Those overlapped oligonucleotides can then be annealed and filled in with Klenow exopolymerase to yield double-stranded sequences. This double-stranded template can then be used in the polymerase chain reaction using either a biotin-labeled 3′-primer or 5′-primer. Accordingly, the double-stranded PCR product will consist of a biotin molecule attached to either the sense or antisense strand of the PCR product. These amplification products can then be heat denatured and quickly cooled to generate a mixture of the two single strands of the double-stranded PCR product. Then, streptavidin-coated magnetic microbeads can be added to the mixture to selectively bind the biotin-containing single stranded complexes, while the desired single strands are removed from the mixture. At that time, a biotin-labeled PNA-clamp may be added to the isolated single-strands, which can be engineered to contain a target recognition site at their 3′-ends. Accordingly, the PNA-clamp will hybridize to the 3′-end of the isolated single-stranded molecules and then NLS-coated streptavidin molecules can be added to the mix. The streptavidin will bind to the biotin on the PNA-clamp forming an apoptosis-inducing hairpin loop nucleic acid complexed with a PNA-clamp and NLS peptides.

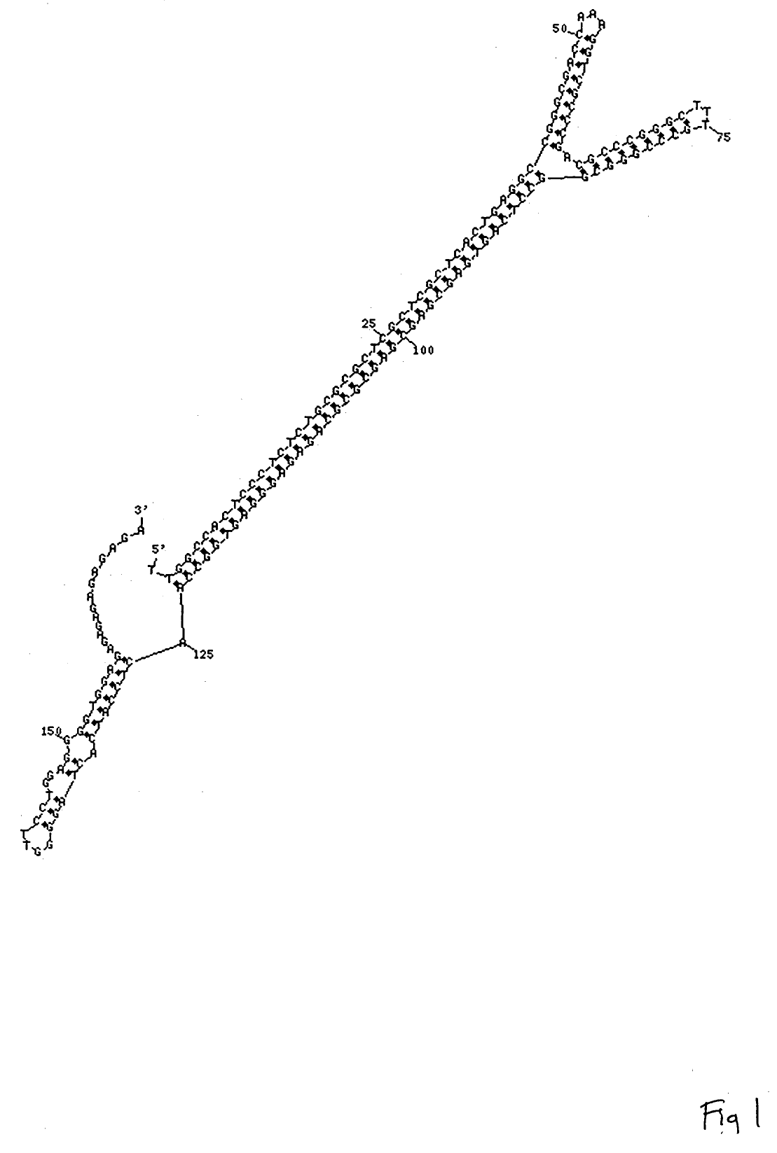

Thus, the present invention envisions the generation of constructs that are capable of folding into the desired multiple “kissing ear” conformation as depicted in FIGS. 1, 2 and 3, by PCR amplification or restriction digestion of plasmids amplified by, and isolated from cell cultures.

The examples described herein are not intended to be limiting and such departures are not outside the scope of the present invention.

What we claim is:

1. An isolated nucleic acid drug comprising four pairs of hairpin loops, wherein each pair of hairpin loops is capable of inducing cell apoptosis.

2. The isolated nucleic acid drug of claim 1, wherein the nucleic acid is DNA.

3. The isolated nucleic acid drug of claim 2, wherein the DNA comprises the sense and antisense polynucleotide sequences of an AAV ITR.

4. The isolated nucleic acid drug of claim 3, wherein said AAV ITR has the sequence described in SEQ ID NO. 1.

5. The isolated nucleic acid drug of claim 1, further comprising at least one nuclear localization signal peptide.

6. The isolated nucleic acid drug of claim 5, wherein said nuclear localization signal peptide is associated with said nucleic acid drug via a PNA-clamp, wherein said PNA-clamp comprises a biotin molecule that is bound to a streptavidin molecule, wherein said streptavidin molecule comprises at least one nuclear localization signal peptide, and wherein said PNA-clamp anneals to a target sequence present in said nucleic acid drug.

7. The isolated nucleic acid drug of claim 5, wherein said nuclear localization signal peptide is selected from the group consisting of an SV40 nuclear localization signal peptide, a poly-L-lysine, an antennapedia peptide, a TAT peptide, a c-myc peptide, a VirD2 peptide, a nucleoplasmin peptide, an ARNT derived peptide and an M9 domain peptide.

8. An apoptosis-inducing formulation comprising a nucleic acid drug which comprises four pairs of hairpin loops.

9. The apoptosis-inducing formulation of claim 8, further comprising a DNase inhibitor.

10. A plasmid comprising a construct that comprises, 5′- to 3′-, (i) a first arm polynucleotide sequence, (ii) a spacer polynucleotide sequence, and (iii) a second arm polynucleotide sequence, wherein said second arm polynucleotide sequence is the complement of said first arm polynucleotide sequence and wherein said second arm polynucleotide sequence is in the opposite orientation of said first arm.

11. The plasmid of claim 10, wherein said construct is flanked by the same or different restriction sites.

12. The plasmid of claim 10, wherein said plasmid comprises at least two of said constructs.

13. The plasmid of claim 10, wherein said plasmid comprises at least four of said constructs.

14. The plasmid of claim 10, wherein said plasmid comprises at least six of said constructs.

15. The plasmid of claim 10, wherein said plasmid comprises at least ten of said constructs.

16. The plasmid of claim 10, wherein said plasmid comprises more than twelve of said constructs.

17. The plasmid of any one of claims 10-16, wherein each construct can be separated from each of the other constructs by exposing said plasmid to one or more restriction enzymes that recognizes the restriction site sequences that flank each of said constructs.

18. A cell comprising the plasmid of any one of claims 10-16.

19. The cell of claim 18, wherein said cell is a bacterial cell, mammalian cell, viral cell, yeast cell or fungal cell.

20. The cell of claim 19, wherein said bacterial cell is an E. coli cell.

21. A nucleic acid drug, comprising (i) a PNA-clamp comprising a biotin molecule; (ii) a streptavidin molecule comprising at least one nuclear localization signal peptide; and (iii) an AAV ITR polynucleotide with a 5′ end and a 3′ end, wherein said PNA-clamp is hybridized to the 3′-end of said AAV ITR polynucleotide, wherein said AAV ITR folds into a pair of hairpin loops, wherein said biotin molecule is bound to said streptavidin molecule, and wherein said nucleic acid drug targets the nucleus or genome of a cell.

22. The nucleic acid drug of claim 21, wherein said nuclear localization signal peptide is selected from the group consisting of an SV40 nuclear localization signal peptide, a poly-L-lysine, an antennapedia peptide, a TAT peptide, a c-myc peptide, a VirD2 peptide, a nucleoplasmin peptide, an ARNT derived peptide and an M9 domain peptide.

23. The nucleic acid drug of claim 21, wherein said AAV ITR polynucleotide comprises the sequence described in SEQ ID NO. 1.

24. A cell comprising the nucleic acid drug of claim 1 or claim 21.

25. The cell of claim 24, wherein said cell is a bacterial cell, mammalian cell, viral cell, yeast cell or fungal cell.

26. The cell of claim 25, wherein said bacterial cell is an E. coli cell.

27. A method for producing a nucleic acid drug comprising, transforming a cell with the plasmid of any one of claims 10-16, incubating the cell under conditions that promote cell growth, isolating the plasmid DNA from the culture, adding at least one restriction enzyme to the isolated plasmid DNA to generate discreet constructs, and denaturing the discreet constructs into single-stranded nucleic acids, wherein the single-stranded nucleic acids hybridize to sequences present in their own strand as well as to complementary sequences in other single strands to produce said nucleic acid drug.

28. The method of claim 27, wherein said cell is a bacterial cell.

29. The method of claim 28, wherein said cell is an E. coli cell.

30. The method of claim 27, further comprising a PNA-clamp comprising a biotin molecule bound to a streptavidin molecule that comprises at least one nuclear localization signal peptide, wherein said PNA-clamp is hybridized to a sequence present in a part of said nucleic acid drug.

31. The method of claim 30, wherein said PNA-clamp is hybridized to a nucleic acid sequence present in the spacer portion of said nucleic acid drug.

32. A method for producing a nucleic acid drug comprising, using the polymerase chain reaction to amplify a polynucleotide sequence that comprises (i) a first arm polynucleotide sequence, (ii) a spacer polynucleotide sequence, and (iii) a second arm polynucleotide sequence, wherein said second arm polynucleotide sequence is the complement of said first arm polynucleotide sequence and is in the opposite orientation of said first arm, isolating the amplification products from said polymerase chain reaction, denaturing the amplification products to form single strands and allowing said single strands to reanneal into hairpin-stem loop structures, wherein at least some of the reannealed structures comprise four pairs of hairpin loops, wherein the reannealed structures are the nucleic acid drugs.

33. The method of claim 32, wherein said first arm polynucleotide sequence has the sequence described in SEQ ID NO. 1, and wherein said second arm polynucleotide is the complement of the sequence described in SEQ ID NO. 1.

34. A method for delivering a nucleic acid drug to the genome of a cell, comprising, providing at least one target cell; and introducing at least one nucleic acid drug of claims 1, 6, 8 or 21 to said target cell, wherein said nucleic acid drug enters said target cell and is directed to the cell nucleus or genome.

35. The method of claim 34, wherein said cell is a eukaryotic or prokaryotic cell.

36. The method of claim 34, wherein said cell is a disease cell.

37. The method of claim 34, wherein said target cell does not contain a functional p53 protein.

38. The method of claim 36, wherein said target cell is a cancer cell.

39. A method for inducing apoptosis in tumor cells of a living animal, comprising, introducing at least one nucleic acid drug of claims 1, 6, 8 or 21 into said animal, wherein said nucleic acid drug enters and targets the genome of cells lacking a functional p53 protein, thereby inducing apoptosis of said cells.

40. The method of claim 39, wherein said nucleic acid drug is introduced into said animal by intravenous injection, topical application, aerosol, through the nasal mucosa, rectally, or orally.

41. The method of claim 39, wherein said animal is a mammal.

42. The method of claim 41, wherein said mammal is a mouse, rat, rabbit, cat, dog, pig, cattle, monkey, or human.

43. The method of claim 42, wherein said mammal is a human.

44. The method of claim 39, wherein said animal is a bird or reptile.


SEQUENCE LISTING (3 sequences)

SEQ ID NO: 1 - DNA, 144 bp, Artificial Sequence
  Description of Artificial Sequence Synthetic AAV ITR nucleotide sequence
ttggccactccctctctgcgcgctcgctcgctcactgaggccgggcgaccaaaggtcgcccgacgccgggctttgcccgggcggcctcagtgagcgagcgagcgcgcagagagggagtggccaactccatcactaggggttcct

SEQ ID NO: 2 - DNA, 25 bp, Artificial Sequence
  Description of Artificial Sequence Synthetic nucleotide target sequence
ggaggggtggagagagagagagaga

SEQ ID NO: 3 - DNA, 170 bp, Artificial Sequence
  Description of Artificial Sequence Synthetic AAV ITR nucleotide sequence
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagagagagagagaga